(19) United States
(12) Patent Application Publication
(10) Pub. No.: US 2012/0270302 A1
(43) Pub. Date: Oct. 25, 2012

(54) Method for Making Dehydrated Mycelium Elements and Product Made Thereby

(76) Inventors: Eben Bayer, Troy, NY (US); Gavin McIntyre, Troy, NY (US)

(21) Appl. No.: 13/454,856

(22) Filed: Apr. 24, 2012

Related U.S. Application Data
(60) Provisional application No. 61/517,749, filed on Apr. 25, 2011.

(51) Int. Cl. C12N 1/14

(52) U.S. Cl. 435/254.1

(57) ABSTRACT

A living hydrated mycelium composite containing at least one of a combination of mycelium and fibers, mycelium and particles, and mycelium, particles and fibers is processed with a nutrient material to promote mycelia tissue growth; thereafter dehydrated to a moisture content of less than 50% by weight to deactivate the further growth of mycelia tissue; and then stored in the form of pellets. The stored may thereafter be re-hydrated and molded or cast into panels that can be separated into cubes or bricks that can be stacked and re-hydrated for making fabricated sections.

This application claims the benefit of Provisional Patent Application 61/517,749 filed Apr. 25, 2011.

This invention relates to a method for making dehydrated mycelium elements and a product made thereby.

As is known from published United States Patent Application 2008/0145577, use can be made of a fungus to form composite materials by mixing an inoculum including a preselected fungus with discrete particles and a nutrient material capable of being digested by the fungus. It is also known from U.S. Pat. No. 8,001,719 to enclose and grow a fungal primordium in a mold to obtain a mass of fungal tissue in the form of low density chitinous material.

It is an object of this invention to provide an improved method for the production of dehydrated mycelium elements.

It is another object of this invention to produce dehydrated mycelium pellets that can be used as is or can be used to make formed elements.

Briefly, the invention provides a method for producing dehydrated mycelium which can be re-hydrated and rapidly re-formed into many different shapes, such as bricks, blocks, pellets and the like elements wherein the adhesion of the elements is achieved through re-animation of a fungal organism which grows the elements together.

In one embodiment, the invention provides a method of making dehydrated mycelium elements comprising the steps of creating a living hydrated mycelium composite containing at least one of a combination of mycelium and fibers, mycelium and particles, and mycelium, particles and fibers; adding a nutrient material to the mycelium composite in an amount to promote mycelia tissue growth; thereafter dehydrating the mycelium composite to a moisture content of less than 50% by weight to deactivate the further growth of mycelia tissue; and thereafter storing the dehydrated mycelium composite at a temperature in the range of from −50° F. to +200° F.

The dehydrated mycelium composite may be processed into a plurality of discrete particles or coated fibers for storage.

The stored dehydrated mycelium composite may then be taken out of storage and further processed by adding moisture to the plurality of discrete particles in an amount sufficient to re-hydrate the particles and to re-activate mycelium on the exterior of the particles for growth into adjacent discrete particles.

Thereafter, the re-hydrated particles may be molded into at least one pellet or molded into at least one aggregated mass with mycelium bonding between and around the particles or re-incubated for a time sufficient for mycelium to bond particles together into a coherent mass.

Thereafter, the coherent mass is dehydrated to a moisture content of from 0 to 30%, at a temperature greater than 150° F. and for a time sufficient to permanently de-activate the mycelium.

In accordance with the method, dehydrated blocks and bricks can be formed which can be milled, cut, or otherwise transformed into new shapes. These shapes when re-hydrated will grow fresh exterior skins, and, when placed in contact, will self adhere to each other.

For example, the re-hydrated particles may first be fabricated into one or more panels, each of which is thereafter separated into a plurality of blocks (or cubes). Moisture is then added to the blocks in an amount sufficient to re-hydrate blocks and to re-activate mycelium on the exterior of blocks for growth into adjacent blocks to bond the blocks together to form a fabricated section. A plurality of such fabricated sections may then be placed in direct contact with each other with mycelium on exterior surfaces of the sections bonding the sections together.

These and other objects and advantages of the invention will become more apparent from the following detailed description taken in conjunction with the accompanying drawings wherein:

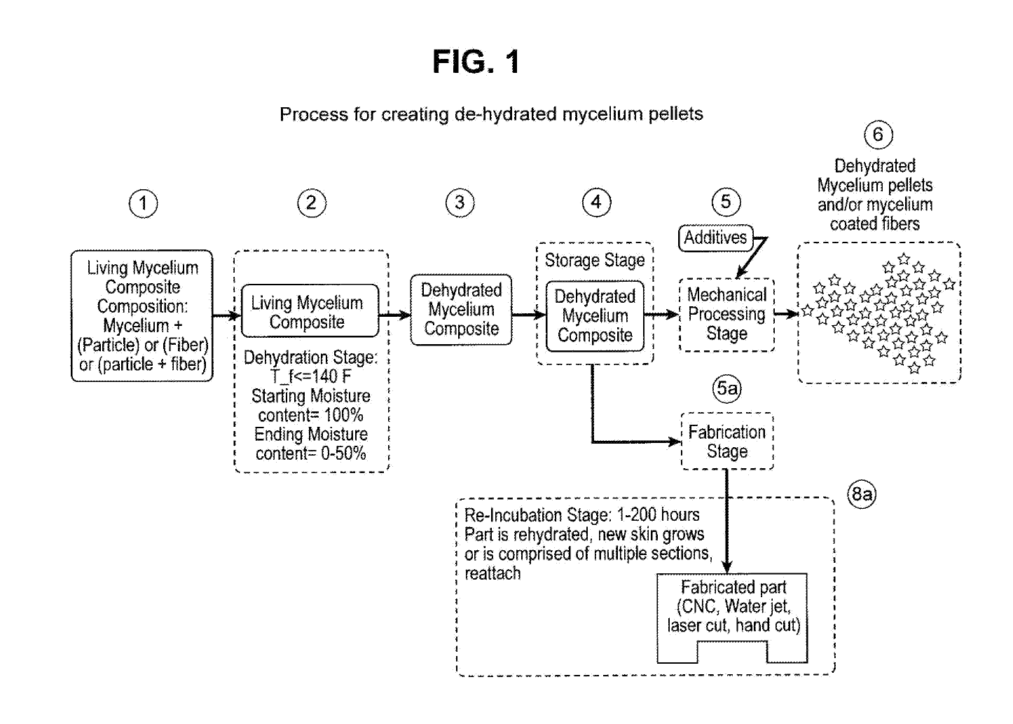

FIG. 1 illustrates initial steps in a method of making mycelium pellets in accordance with the invention;

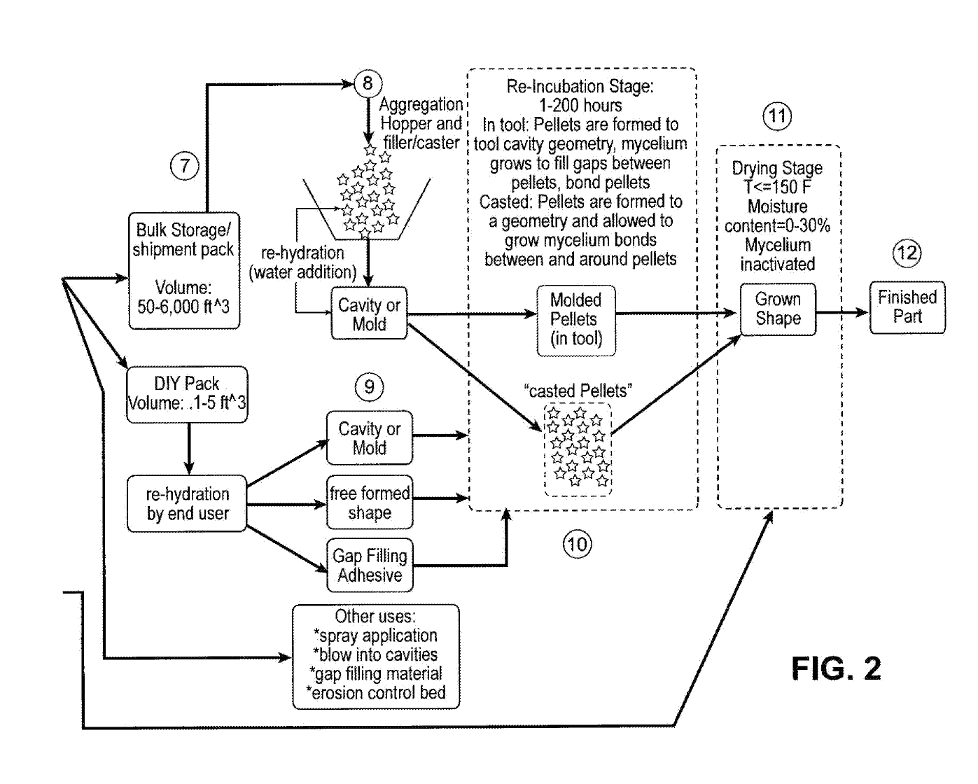

FIG. 2 illustrates further steps in the method of FIG. 1;

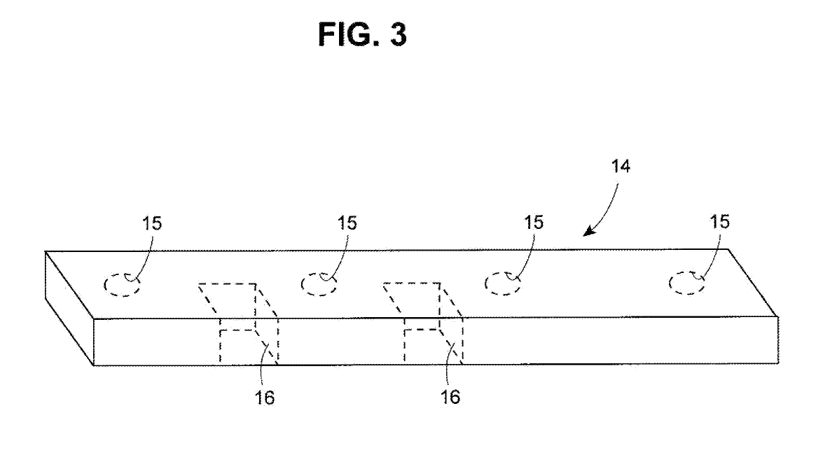

FIG. 3 illustrates a perspective view of a plank formed in accordance with the invention;

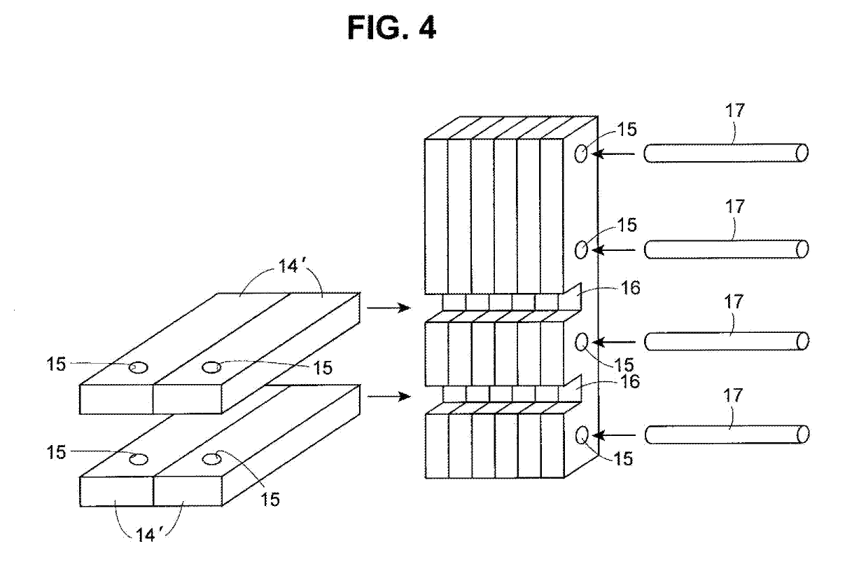

FIG. 4 illustrates an exploded view of a plurality of planks for forming a useful product in accordance with the invention;

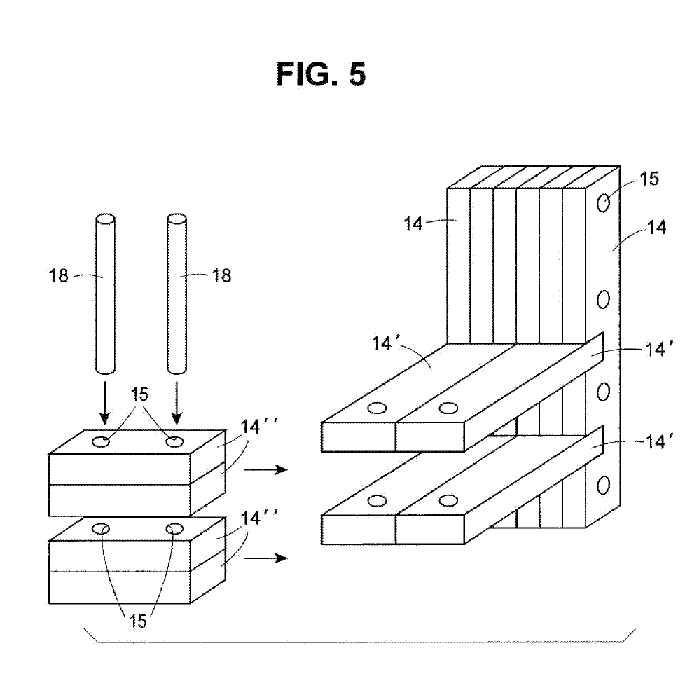

FIG. 5 illustrates an exploded view of additional planks being added to the structure of FIG. 4;

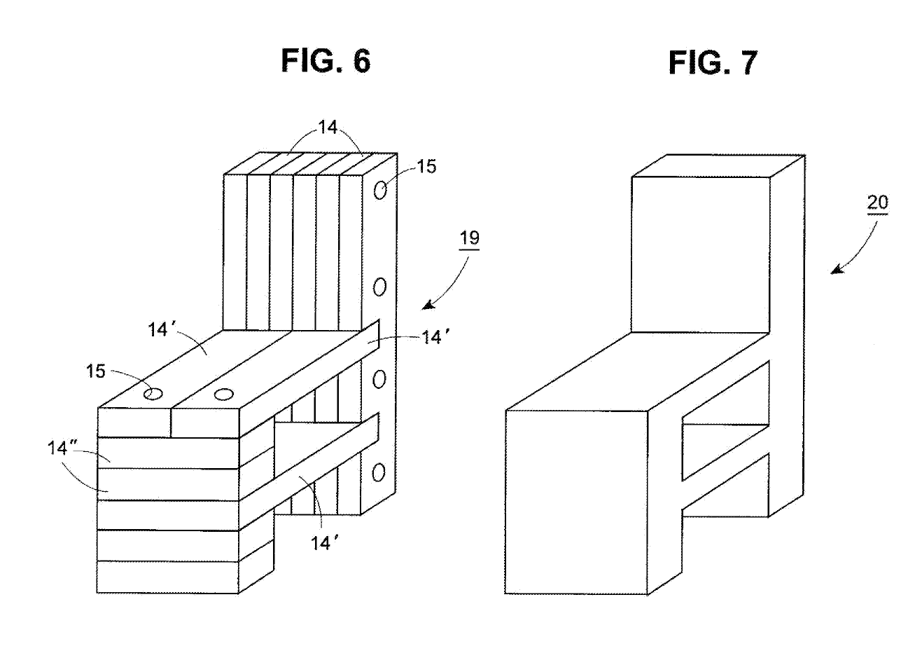

FIG. 6 illustrates a perspective view of an assemblage of planks for forming a useful product prior to hydrating the assemblage; and

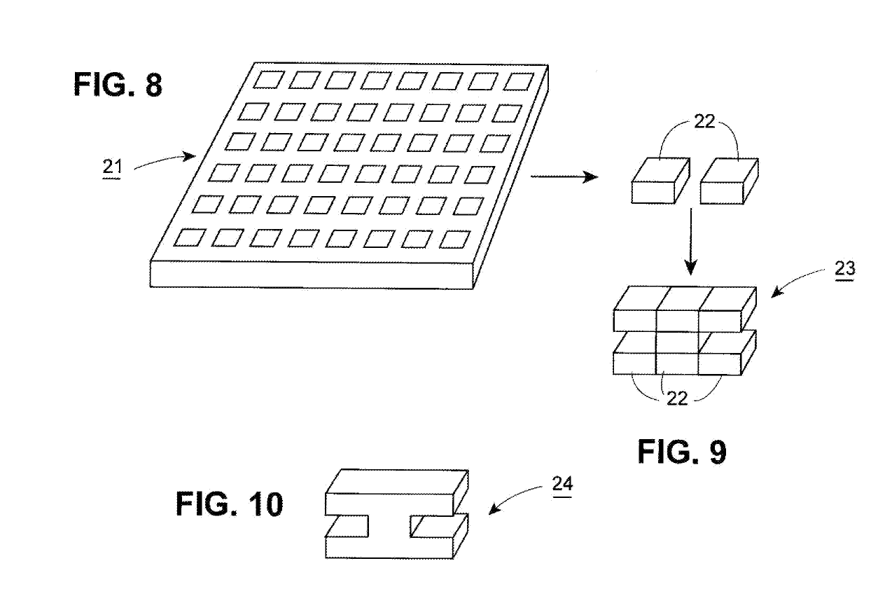

FIG. 7 illustrates a perspective view of the finished product made from the assemblage of FIG. 6.

Referring to FIG. 1, in accordance with a first step 1, a living mycelium composite is created. This can be a combination of mycelium and fibers, mycelium and particles, or both mycelium, particles, and fibers. Generally, a nutrient material is included with mycelium and fibers to promote mycelia tissue growth, such as described in Ser. No. 12/001,556, filed Dec. 12, 2007.

In accordance with step 2, the living composite from step 1 is dehydrated at a temperature less than 140° F. from a starting moisture content of 100% to an ending moisture content of less than 50%. In accordance with step 3, the dehydrated mycelium composite is now in-active, and will no longer grow mycelia fibers, and will be incapable of producing mushrooms, primordia, or other tissue.

In accordance with step 4, the dehydrated mycelium composite is placed in storage stage and stored indefinitely, at temperatures ranging from −50° F. to 200° F. Lower moisture contents allow higher temperature storage ranges.

In accordance with step 5, the dehydrated mycelium composite from step 4 is mechanically processed into a plurality of particles or fibers in a mechanical processing stage. Additional additives, such as nutrients, grown enhancing compounds, binding agents, addition particles, additional fibers, or other materials may be added at this stage.

In accordance with step 6, the resulting particles and/or fibers geometry and size can be tuned to result in different densities and self adhesion characteristics. In the case of fibers, the fibers appear as being coated with mycelium.

Referring to FIG. 2, in accordance with step 7, the dehydrated mycelium composite particles and/or fibers from step 5 are stored in bulk, for example, in volumes of from 50 to 6000 cubic feet in step 7 or packed into a variety of containers or transport vessels for distribution including bulk shipments, super-sacks, tractor trailers, or small DIY packages of from 1 to 5 cubic feet (e.g. in 1 to 10 gallon containers).

Also, the dehydrated mycelium composite particles and/or fibers from step 5 may be used in other applications, such as for spray application, or by being blown into cavities and/or as gap filling material and in erosion control beds.

After storage and/or shipment, e.g. to a point of use, the dehydrated mycelium composite particles and/or fibers from step 7 are re-hydrated in accordance with step 8 to allow growth through the addition of moisture. Moisture can be added such that particles return to a 100% moisture, or just enough moisture can be added (often less than 10% of the starting volume) such that the mycelium on the exterior of the particles re-activities and is able to grow into adjacent particles.

In accordance with step 8, the dehydrated mycelium composite particles and/or fibers are delivered into an aggregation hopper and filler caster with water being added to the hopper, for example, by spraying. The re-hydrated mycelium composite particles and/or fibers are then delivered into a cavity or mold to be molded into elements, such as pellets.

In a similar manner, the dehydrated mycelium composite particles and/or fibers that are packaged in the small DIY packages can be rehydrated in a modified step 9 by the end user in any suitable manner and then placed in a cavity or mold to form an aggregated mass, or used as a free form shape or have an adhesive added to fill the gaps between the particles and/or fibers.

In accordance with step 10, the elements from step 9 are subject to a re-incubation stage for a period of time of from 1 to 200 hours in either a tool or are casted.

For example, the molded pellets from the cavity or mold of step 9 are formed to a tool cavity geometry and the mycelium allowed to grow to fill the gaps between the pellets. Alternatively, the pellets are formed to a geometry and allowed to grow mycelium bonds between the pellets.

In a similar manner, the pellets or elements formed from the dehydrated mycelium composite particles and/or fibers that are packaged in the small DIY packages are allowed to incubate and grow together in the re-incubation stage or may be casted into a free supporting form. An additive may be applied to promote initial adhesion, or may be added at stage 5.

In accordance with step 11, the grown shape from the re-incubation stage is once again dehydrated in a drying stage to a moisture content of from 0 to 30%, at a temperature greater than 150° F., such that the mycelium is permanently inactivated.

Thereafter, the resulting dehydrated shape is delivered in accordance with step 12 as a finished part suitable for use.

Alternately, in accordance with step 5a, the dehydrated mycelium composite from step 4 may be fabricated in a fabrication stage into panels and the dehydrated panels may be cut, for example, by CNC, water jet, laser cut, hand cut and the like, or otherwise transformed into a fabricated bulk shape, such as into blocks or bricks.

Thereafter, in accordance with step 8a, the mycelium composite fabricated blocks are subjected to a re-incubation stage wherein the blocks are rehydrated to grow fresh mycelium around joints that have been cut. As above described, re-incubation takes place over a time of from 1 to 200 hours whereby a part is rehydrated, new skin grows or, if comprised of multiple sections, reattach.

In addition, multiple fabricated sections may be placed in direct contact to promote growth adhesion between sections.

The entire block may be hydrated, or the exterior surfaces may be hydrated, both will promote exterior mycelia growth. The hydrated blocks are then dried as in step 11 and delivered as in step 12 as finished products.

Referring to FIG. 3, a finished part 14 produced in accordance with the above-described method may be in the form of a dehydrated block 14 (hereinafter “mycoblock”). Such a mycoblock 14 may be drilled to form bores 15 extending therethrough at predetermined places and otherwise cut or machined to form recesses 16 at predetermined places.

Referring to FIG. 4, in order to form a useful product, a plurality of mycoblocks 14, e.g. six, are stacked with the bores 15 in alignment and dowels 17 are passed into the aligned bores 15 to hold the mycoblocks 14 together. In addition, additional mycoboards 14′ of a shorter length are placed together in pairs and inserted into the recesses 16 of the stacked mycoboards 14. As illustrated, each of the shorter mycoboards 14″ has a bore 15 at an end opposite the end that is fitted into the stacked mycoboards 14.

Referring to FIG. 5, in a further step, additional mycoboards 14″ of even shorter length than the mycoboards 14′, each with a pair of bores 15 are placed together in pairs and inserted between the free ends of the mycoboards 14′ extending from the stacked mycoboards 14. In addition, dowels 18 are inserted in the bores 15 of the mycoboards 14″ to hold them to the free ends of the mycoboards 14′ and to complete an assemblage.

Referring to FIG. 6, the assemblage 19 of mycoboards 14, 14′,14″ is then subjected to rehydration wherein water is added to the assemblage to reanimate the fungus and the mycoboards bound into a cohesive unitary structure.

Referring to FIG. 7, the rehydrated assemblage 19 is allowed to incubate for a time, such as from 3 to 5 days, sufficient to form a self-supporting useful product 20 and is then dried, for example in sunlight, to inactivate the fungus.

The method described above may be summarized as making grown mycological composites, desiccating the materials, processing to create a uniform particle, and then reanimating the materials to create a new uniform solid.

The following presents a more detailed description of one embodiment of the method.

### Production of the Tissue Culture

A solid grain carrier was used as the predominate substrate for inoculation. The grain spawn was grown and prepared in batches of five gallons of dedicated grain, spawn on a weekly basis. Each gallon of grain spawn was comprised of 800 g of yellow millet, 600 mL of de-ionized water, and 10 g of gypsum. Bags of the grain spawn were sterilized in an autoclave at 240° F. and 15 psi for one hour and then allowed to cool to room temperature in a HEPA filtered laminar flow hood.

The bags of grain were then inoculated with the mycelium culture (tenth of a 100 mm Petri dish culture per bag) and incubated in ambient lab conditions and full colonization was achieved in five to seven days.

### Determination of Optimal Method for Particle Production

The first iteration of this task used 20 L of each substrate, fiberized cotton gin waste and oat hulls, that were sterilized using an autoclave and then inoculated with grain spawn.

The bags were inoculated in a HEPA filtered laminar flow hood at a 20% [m:m] rate, and then incubated until colonization was complete.

Once colonization was achieved the materials were desiccated in a laminar flow hood at ambient conditions over the course of four days. The materials were separated bagged and labeled and sent to the USDA Agricultural Research Service in Lubbock, Tex., for machine processing. The process equipment included a hammer mill (0.25″ and 1″ screen sizes), a rotary shear cutter (dual axis with helical cutters), screen-classified cutter (single axis with knife edges at the edge of the hopper), and a Titan shear (dual axis with cutters mounted on the shafts, rotating inward).

The results from a first iteration found that the dehydrated blocks processed in the hammer mill offered the best mycelium reanimation with the least residual contamination when incubated on an agarose media (MEA). These blocks, however, were not uniform in moisture content due to the amorphous form factor and as such this portion of the study was repeated using the same methods and equipment.

### Particle Moisture Content and Viability

In determining moisture content, 15 L of each substrate type was sterilized in an autoclave and then inoculated with Ganoderma resinaceum grain spawn. The inoculated substrate was evenly divided by mass into 18 tools per substrate (12″×5″×1″). All replicates, 36 total, were incubated until colonization was complete after seven days.

Each set of 18 was divided into three sets of six, representing three drying temperatures (i.e. 24° C., 53° C., and 82° C.). The materials desiccated at room temperature were placed in a HEPA filtered laminar flow hood (forced convection), while the other two sets were dried in a programmed Despatch Convection Dryer that is positively pressurized with HEPA filtered air.

Each set of six was then measured for moisture content on an hourly basis using a conductivity meter, until two replicates each achieved 10%, 30%, and 50% moisture content by weight.

Once the drying cycles were complete each of the 18 sets were evenly divided by mass into six separate storage bags, representing six months of the reanimation study. The dehydrated particles were then placed on PDA and TSA, which are selective for molds and bacteria selectively. For the fiberized cotton burr particles the least contamination was observed on the sets dried at 82° C., including no bacteria contamination, and the best recovery was found with the materials desiccated at ambient temperatures. The oat hull sets had less contamination and better growth over all when compared to the fiberized cotton burr. The best desiccation level initially appears to be 30% moisture with a drying temperature around 53° C.

The particle viability study sought to determine the optimal storage time for the desiccated particles based on dehydration temperature, stored moisture content, and substrate. The two primary sets were divided into sterilized substrate and plant essential oil disinfected substrate. The two sets were analyzed over the course of six months measuring conducting a binary growth analysis, contamination observations, and radial growth over after five days of incubation from the inoculation point. All six months of the sterilized sets have been completed and the plant essential oil sets have been completed for the first four months.

The optimal moisture content and drying temperature for storage is 10% at 53° C. for all sets with the exception of the fiberized burr substrate disinfected with a plant essential oil emulsion. The 50% moisture sets predominately harbors microbial contaminates, such as bacteria and mold, and as such should not be used for long-term storage. The materials disinfected with the plant essential oils have a better range of dehydration temperatures and moisture contents for storage, since more of the sets exhibited reanimation.

The invention thus provides a relatively simple and economic method of making dehydrated mycelium elements that can be used as is or in the subsequent fabrication of various shaped parts.

The dehydrated block of the invention which may be re-hydrated is distinguished from a dehydrated block of the invention that is not capable of being re-hydrated in that the block which may be re-hydrated is characterized in having a moisture content of less than 30% and in having the mycelium therein in a de-activated state capable of being re-activated for growth upon the addition of moisture whereas a dehydrated block of the invention that is not capable of being re-hydrated is characterized in having the mycelium in a permanently de-activated state that is not capable of being re-activated for growth.

What is claimed is:

1. A method of making dehydrated mycelium elements comprising the steps of
creating a living hydrated mycelium composite containing at least one of a combination of mycelium and fibers, mycelium and particles, and mycelium, particles and fibers;
adding a nutrient material to said mycelium composite in an amount to promote mycelia tissue growth;
thereafter dehydrating the mycelium composite to a moisture content of less than 50% by weight to deactivate the further growth of mycelia tissue; and
thereafter storing the dehydrated mycelium composite at a temperature in the range of from −50° F. to +200° F.

2. A method as set forth in claim 1 further comprising the step of processing the dehydrated mycelium composite into a plurality of discrete particles.

3. A method as set forth in claim 2 further comprising the step of adding an additive to said plurality of discrete particles selected from the group consisting of nutrients, grown enhancing compounds, binding agents, additional particles and additional fibers.

4. A method as set forth in claim 1 further comprising the step of adding moisture to said plurality of discrete particles in an amount sufficient to re-hydrate said discrete particles and to re-activate mycelium on the exterior of said discrete particles for growth into adjacent discrete particles.

5. A method as set forth in claim 4 wherein said moisture is added to said plurality of discrete particles in the form of a spray of water.

6. A method as set forth in claim 4 further comprising the step of molding said re-hydrated discrete particles into at least one pellet.

7. A method as set forth in claim 4 further comprising the step of molding said re-hydrated discrete particles into at least one aggregated mass of particles with mycelium bonding between and around said particles.

8. A method as set forth in claim 4 wherein said re-hydrated discrete particles are re-incubated for a time sufficient for mycelium to bond said particles together into a coherent mass.

9. A method as set forth in claim 8 wherein said time is from 1 to 200 hours.

10. A method as set forth in claim 8 further comprising the step of thereafter dehydrating said mass to a moisture content of from 0 to 30%, at a temperature greater than 150° F. and for a time sufficient to permanently de-activate the mycelium.

11. A method as set forth in claim 2 further comprising the steps of fabricating said plurality of discrete particles into at least one panel and thereafter separating at least one of said panels into a plurality of blocks.

12. A method as set forth in claim 11 further comprising the step of adding moisture to said plurality of blocks in an amount sufficient to re-hydrate said blocks and to re-activate mycelium on the exterior of said blocks for growth into adjacent blocks of said plurality of blocks to bond said blocks together to form a fabricated section.

13. A method as set forth in claim 12 wherein a plurality of said fabricated sections are placed in direct contact with each other with mycelium on exterior surfaces of said sections bonding said sections together.

14. A fabricated section comprising a plurality of blocks, each said block being made of mycelium composite containing at least one of a combination of mycelium and fibers, mycelium and particles, and mycelium, particles and fibers, and each said block being bonded to an adjacent block to form a cohesive unitary structure.

15. A fabricated section comprising a plurality of blocks, each said block being of cubic shape made of mycelium composite containing at least one of a combination of mycelium and fibers, mycelium and particles, and mycelium, particles and fibers, and each said block being bonded to an adjacent block to form a cohesive unitary structure.

16. A fabricated section as set forth in claim 15 wherein said cohesive unitary structure is of three dimensional H-shape.

17. A dehydrated block having a predetermined shape and being made of mycelium composite containing at least one of a combination of mycelium and fibers, mycelium and particles, and mycelium, particles and fibers, said block being characterized in having a moisture content of less than 30% and in having said mycelium therein in a de-activated state capable of being re-activated for growth upon the addition of moisture.

18. A block for forming a fabricated section, said block having a predetermined shape and being made of mycelium composite containing at least one of a combination of mycelium and fibers, mycelium and particles, and mycelium, particles and fibers, said block having a plurality of bores passing therethrough for receiving an alignment dowel.

19. A block for forming a fabricated section, said block having a predetermined cubic shape and being made of mycelium composite containing at least one of a combination of mycelium and fibers, mycelium and particles, and mycelium, particles and fibers.
